(19) United States
(12) Patent Application Publication
(10) Pub. No.: US 2007/0191751 A1
(43) Pub. Date: Aug. 16, 2007

(54) Rotator cuff, and other shoulder-related, traction relief

(76) Inventor: Ron H. Ruff, Tualatin, OR (US)

Correspondence Address: ROBERT D. VARITZ, P.C., 4915 SE 33RD PLACE, PORTLAND, OR, 97202, US

(21) Appl. No.: 11/250,918

(22) Filed: Oct. 14, 2005

Related U.S. Application Data
(60) Provisional application No. 60/626,704, filed on Nov. 9, 2004.

(51) Int. Cl. A61F 5/00

(52) U.S. Cl. 602/36

(57) ABSTRACT

A method for addressing shoulder issues involving each of (a) a torn rotator cuff, and (b) a post-shoulder-surgery condition, featuring applying to the region of such a rotator cuff/condition, via the associated humerus, an ambulatory, non-gravity-based, downwardly directed, and, where desired, a laterally outwardly directed, elastic traction force. Apparatus for implementing this methodology includes (a) a subject-wearable, passive, elongate, yieldable-resistance, force-applying device possessing opposite ends, and between those ends, a line of action which, with the apparatus in place relative to a subject and to the subject's shoulder and rotator cuff/condition, lies generally parallel to the long axis of the subject's associated humerus, and (b) structure associated with these opposite ends for anchoring them operatively to spaced points on the subject's anatomy in a manner whereby the force-applying device applies to the anatomy a force which tends to produce separation between the head of the humerus and the rotator cuff.

### CROSS REFERENCE TO RELATED APPLICATION

This application claims priority filing-date benefit to currently pending U.S. Provisional Patent Application Ser. No. 60/626,704, filed Nov. 9, 2004, for “Rotator Cuff Traction Relief”. The entire disclosure content of that prior-filed provisional application is hereby incorporated herein by reference.

### BACKGROUND AND SUMMARY OF THE INVENTION

This invention relates to providing pain relief and healing promotion in relation to what is known as a rotator cuff tear, and in particular, pertains to methodology and to apparatus for implementing anatomical displacement compression relief in the region of a rotator cuff tear.

The invention also relates to other shoulder-relief issues, such as a condition involving shoulder discomfort which requires relief and healing after a shoulder surgery. The invention is described herein, for illustration purposes, in the context principally of a rotator cuff tear, but it should be understood that it is fully applicable to the issues of pain relief and healing relating to a post-shoulder-surgery condition. It should thus be fully understood that specific reference herein made to a rotator cuff tear is also intended to be reference to the mentioned post-surgery condition. Accordingly, this “co-focus”situation for the invention is sometimes referred to herein with the expression “rotator cuff/condition”.

Those who are familiar, from first-hand experience, with an unhealed rotator cuff tear understand that, during normal daytime hours, when the body is more or less in an upright condition, gravity, which acts on the particular arm associated with the torn rotator cuff produces a sufficient downward pull with respect to the region of the injured cuff usually to take enough pressure (compression) away from that part of the anatomy effectively to minimize cuff tear pain. However, at night, and under other circumstances when a person is lying down, gravity no longer provides such a decompressing force, and it is very well known that, in this condition, one who has a rotator cuff tear can experience a significant amount of pain, and often a relatively serious sleep-depriving pain.

In the setting of prior art approaches aimed at addressing this kind of an injury condition, it is typical that some form of relatively simple shoulder-area immobilization, linked often with various considered-to-be appropriate surgical procedures, become employed as main approaches toward trying to resolve and heal a rotator-cuff tear. While these and related approaches may, and in certain instances certainly do, provide pain relief and eventual healing promotion, a rotator cuff tear injury continues to present itself as a quite difficult-to-resolve problem for people.

The present invention is based upon the discovery of what is believed to be an entirely unique approach in the healing art for dealing with a rotator cuff tear, and to this end, proposes unique and very effective methodology and apparatus for implementing that methodology directed toward addressing such a medical issue.

Very specifically, the methodology and apparatus of the present invention, which, as will be explained and illustrated below and herein, take a number of different useful forms, are based upon the discovery that rotator cuff tear pain and healing relief can be addressed extremely successfully. Specifically, the invention “acts” through applying certain forces, typically through the humerus, to decompress the region adjacent a rotator cuff tear, independent of gravity action, and in a condition which can be considered to be entirely ambulatory, in the sense that the methodology of the invention can be employed not only while a person is lying and asleep at night, but also when that person is up and around during the daytime. In particular, a principal decompression force which is implemented by the present invention is a downward force basically directed downwardly (through the humerus) along the side of a person's body under circumstances with the arm which is associated with a rotator cuff injury cocked at an angle at the elbow, and with the lower arm suspended and supported in a relatively conventional sling arrangement. Downward decompression force in the region of the rotator cuff may be implemented either through a tension device which preferably acts between the region of the elbow and a lower anatomical region such as the thigh region, or by a compression device which acts between the lower arm and the underarm along the length of the humerus.

Yet another part of the discovery which lies at the core of the present invention involves the application of a lateral force which acts between the side of the body and the upper arm, further to decompress the region adjacent a rotator cuff tear through creating a stabilized, lateral outwardly directed vector, or force, which produces a certain amount of laterally outwardly directed decompression in the subject injury area.

A further discovery which contributes an underpinning to the present invention is that considerable pain relief and healing promotion can be accomplished by simultaneously applying both of these kinds of forces, whereby downward and laterally outwardly directed force vectors produce both lateral and vertical downward and outward decompression in the region of a torn rotator cuff.

Several forms of apparatus useful for implementing these decompression methodologies are illustrated and described herein, and all of the features of the present invention will become more fully apparent as the description which now follow is read in conjunction with the accompanying drawings.

### DESCRIPTION OF THE DRAWINGS

FIG. 1 illustrates, in a fragmentary, skeletal-form manner, the internal structure of a person's right shoulder, and specifically that region of the shoulder which contains the rotator cuff.

FIG. 2 is a fragmentary view, much like that presented in FIG. 1, but here showing the rotator cuff which has an illustrated tear injury.

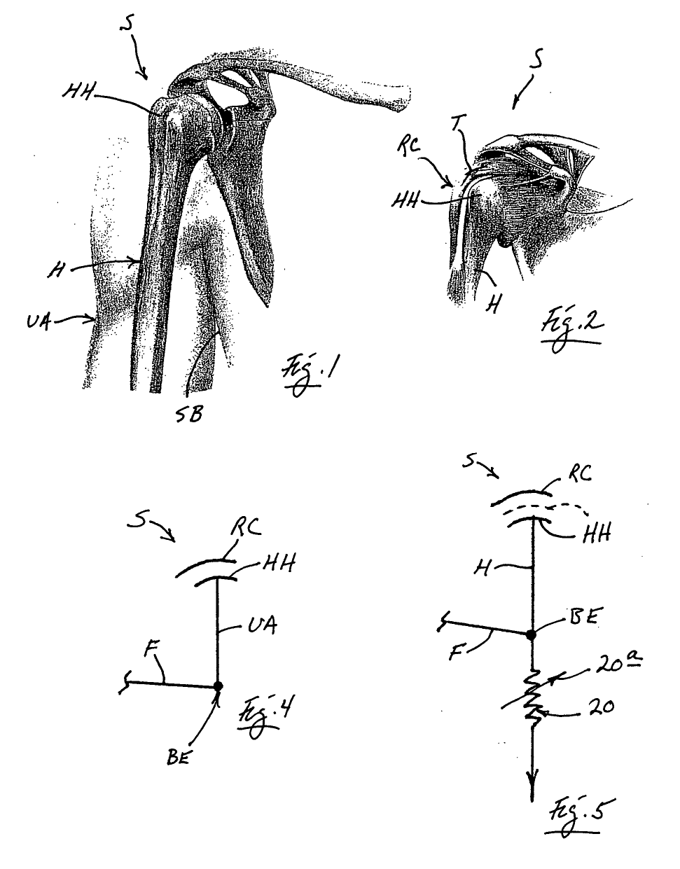

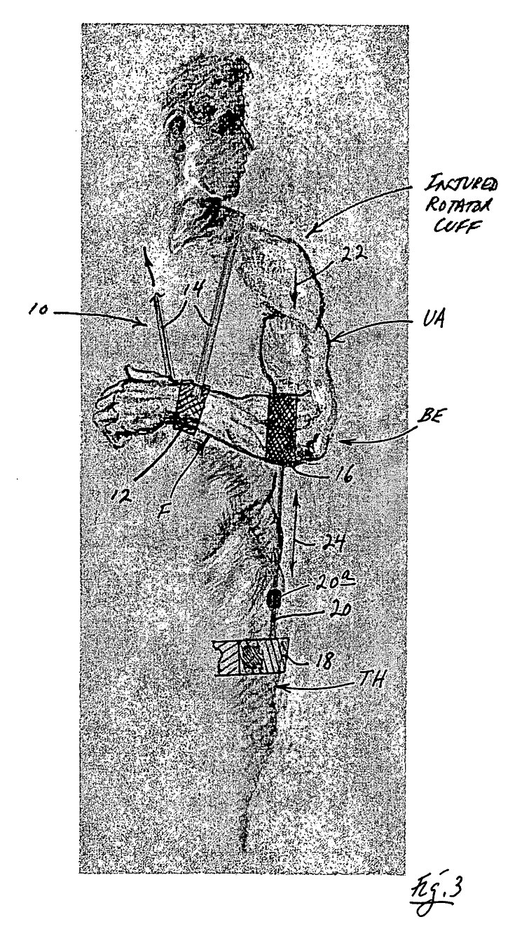

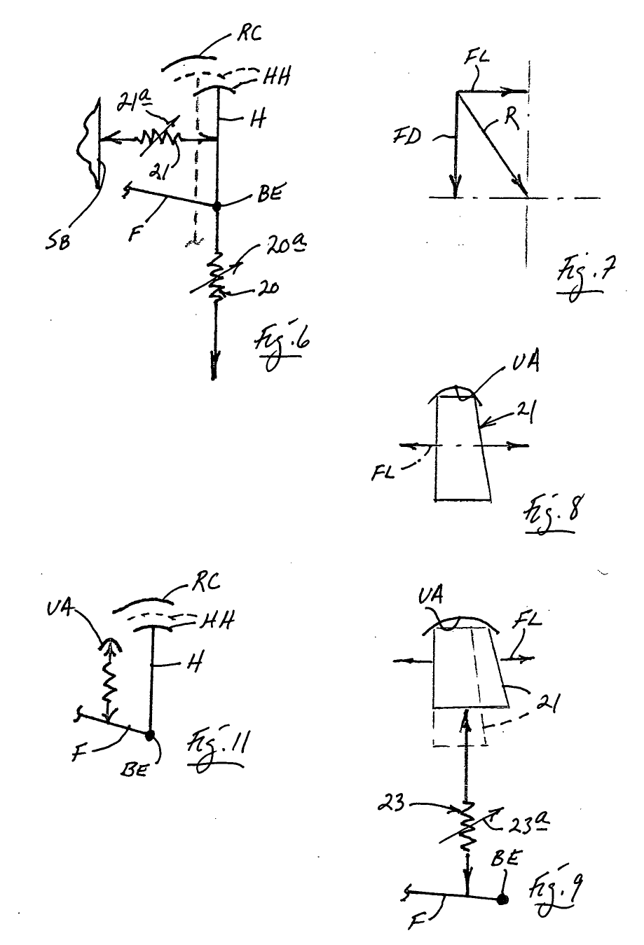

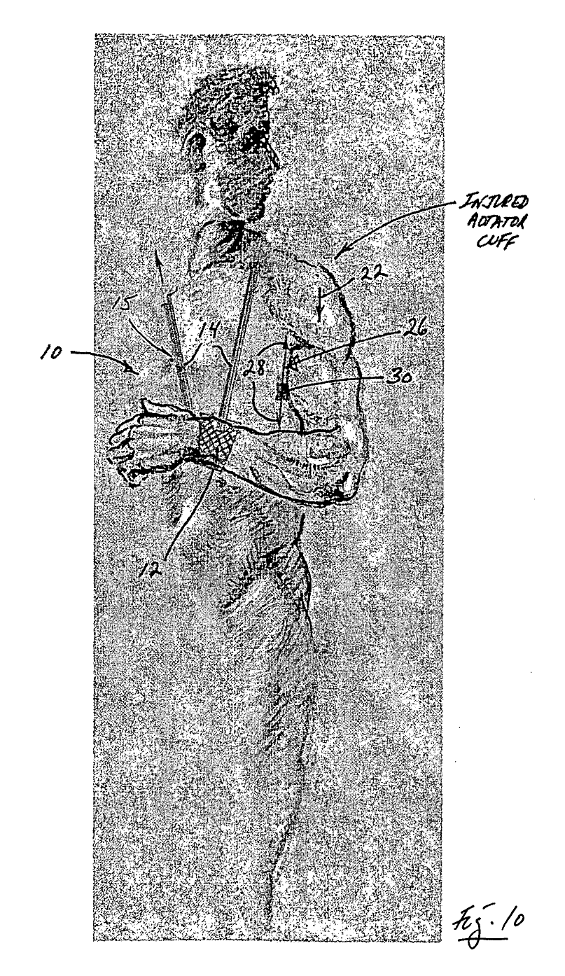

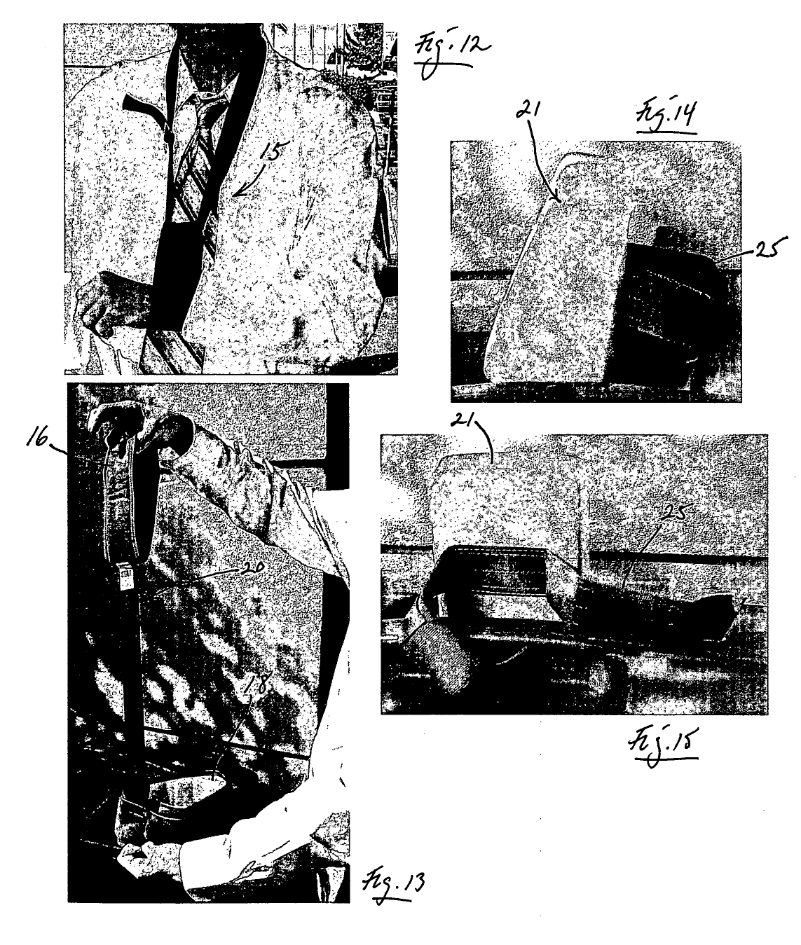

FIGS. 1 and 2 are modified versions of illustrations presented on page 22 in “The World's Best Anatomical Charts”, Third Edition, ISBN 0-9603730-5-5.

FIG. 3, which is on the second plate of the drawings, generally illustrates one form of apparatus which implements one manner of practicing the methodology of the present invention.

FIGS. 4 and 5 are high-level, fragmentary, and very simplified, schematic views generally illustrating practice of the invention in accordance with the apparatus and the methodology illustrated in and associated with FIG. 3.

FIG. 6 is a view somewhat like that presented in FIG. 5, but here showing a slightly modified form and practice of the invention relative to that which is illustrated in FIGS. 3-5, inclusive.

FIG. 7 is a vector diagram illustrating forces that are applied and in existence in accordance with the modified form of the invention shown in FIG. 6.

FIGS. 8 and 9 are high-level, fragmentary, schematic, and very simplified, diagrams illustrating two manners of practicing lateral force application, as well as vertical downward force application (somewhat as is pictured in FIGS. 6 and 7).

FIG. 10 is a view which is similar to FIG. 3, with FIG. 10 showing very schematically yet another modified form and practice of the present invention.

FIG. 11 is a high-level, fragmentary, schematic, and very simplified view generally illustrating practice of the invention as such is pictured in FIG. 10.

FIG. 12 is a photograph showing an arm-supporting sling like that pictured in FIGS. 3 and 10.

FIG. 13 is a photograph illustrating an isolated, wearable tension-applying device such as that shown schematically in FIG. 3.

FIGS. 14 and 15 are photographs showing, from two different points of view, an isolated lateral force-applying device such as the one pictured schematically in FIGS. 8-10, inclusive.

### DETAILED DESCRIPTION OF THE INVENTION

Turning attention first of all to FIGS. 1 and 2, here, certain bone, skin and muscle tissues which are relevant to the discussion and illustration of the practice of the present invention are illustrated. FIG. 1 shows at H the humerus having the usual humeral head HH which is disposed in the region of the shoulder S, with the associated side of the body being shown at SB, and the upper portion of the upper arm structure being shown at UA. FIG. 2 adds imagery which shows the rotator cuff RC, and a tear injury T in this rotator cuff.

In accordance with implementation of the present invention, one approach for dealing with this illustrated rotator cuff injury is to apply, effectively, a downward traction force on the humerus for the purpose of decompressing vertically the region directly associated with the rotator cuff tear.

FIG. 3 in the drawings illustrates one preferred from of apparatus which implements this manner of practicing the present invention. Whereas FIGS. 1 and 2 in the drawings picture a region of a subject's right shoulder, FIG. 3 illustrates a subject's left shoulder in which the region of a rotator cuff injury, such a the tear injury illustrated in FIG. 2, is clearly word-labeled in FIG. 3.

As can be seen very clearly in FIG. 3, and in accordance with the manner of practicing the invention specifically shown in this figure, the subject's left arm, which is associated with the left shoulder wherein a rotator cuff tear exists, is preferably bent at the elbow to create a bent-elbow condition as shown at BE in FIG. 3. In this condition, the upper arm and the forearm F are disposed generally at a right angle relative to one another, with the forearm being supported in this condition through a wrist band 12 and a neck strap 14 which collectively form a sling 15. Sling 15 is also pictured in the photograph of FIG. 12 Band 12 and strap 14 effectively form portions of the overall apparatus 10 which is constructed in accordance with one preferred embodiment of the invention to implement the practice pictured in FIG. 3. Such a condition, namely the bent elbow condition, is established during times that the apparatus of the invention is in place to provide relief for the injured rotator cuff, and this might be done at any time typically during the day when the injured subject decides to make use of the invention.

With the subject's arms so bent, and looking here also at FIG. 13, suitable additional bands (such as Velcro®-closure bands) 16, 18 are attached, respectively, to the forearm adjacent the bent elbow, and to the thigh, TG, which is directly below. An elongate tension element 20, also referred to herein as an elongate yieldable-resistance structure, has its opposite ends appropriately secured to bands 16, 18 so as to be tensed, and to provide and apply a downward pull, represented by double headed arrow 24, on the arm and through the humerus in the upper arm, thus to establish a condition of vertical decompression in the region of the rotator cuff tear. The humerus is thus placed by this apparatus in a traction condition which tends to draw downwardly as indicated by arrow 22 on the region of the rotator cuff to provide useful compression relief on the rotator cuff tear per se.

While many different types of tension element 20 may be employed, one very convenient and effective form for such an element is an elongate elastomeric strap having an adjustable buckle structure, such as that shown at schematically at 20a in FIG. 3, by way of which the amount of downward decompression force transmitted to the arm may be varied.

With attention directed for a moment to FIGS. 4 and 5, it will be seen that these two drawing figures provide a useful schematic way of visualizing practice of the invention as pictured in FIG. 3.

FIG. 4 illustrates generally the condition of the anatomy immediately associated with the rotator cuff before any downward decompression traction force is applied by device 20.

FIG. 5 illustrates changes which take place when device 20 is properly operatively disposed between the bent elbow and the thigh and adjusted appropriately to produce a downward decompression force which tends to shift the humerus and humeral head downwardly away from the rotator cuff, thus to decompress the region immediately associated with the rotator cuff. In FIG. 5, the dashed lines illustrate the position of the humeral head before the application of downward force, and solid lines illustrates a downwardly moved position for the humeral head after downward force application.

Turning attention now to FIGS. 6 and 7, FIG. 6 illustrates a modified version of what is shown in FIG. 5, with this modified version including, in addition to tension device 20, a compression device 21 which applies a lateral separation force effectively between the side of the body SB and (through the upper arm) the humerus H. Dashed lines in FIG. 6 illustrate anatomical conditions before the application of any decompression force to the region of the rotator cuff, and solid lines illustrate conditions after the applications of both downward and lateral separation forces.

FIG. 7 is a vector diagram illustrating the downward force FD the lateral separation force FL and resultant vector force R.

Thus one can see that under the circumstances illustrated in FIGS. 6 and 7, decompression takes place both vertically downwardly and laterally outwardly. Compression device 21 may be of any suitable form, and is preferably made to be force adjustable through a suitable adjusting mechanism shown schematically at 21a in FIG. 6.

FIGS. 8 and 9, along with the photographs presented in FIGS. 14 and 15, illustrate one form of a compression device 21 which has been found to provide a very workable implementation of that form of the present invention which includes both lateral outward and downward decompression forces. More specifically, as illustrated in FIGS. 8 and 9, compression device 21 takes the form a suitable block of a resilient, compressible foam made of any suitable compressible foam material and preferably possessing a front-view outline such as that pictured especially in FIGS. 8 and 14. This block of material is preferably placed between the upper arm and the side of the body immediately under the underarm, and may be held in place through a suitable Velcro®-closure strap, such as that shown at 25 in FIGS. 14 and 15, which binds it in place to an appropriate location on and along the upper arm. With such a block of material in place, when the bent elbow condition is created with the forearm, and is stabilized in a sling, a downward decompression force is exerted through a tension device, such as previously described device 20, the block of material which forms device 21 compresses slightly to apply a lateral separation force FL.

Adjustability of the amount of applied lateral separation force can be accomplished in a number of different ways as, for example, by supplying blocks of material having different dimensions, as well as by modifying and thus changing the downward force produced by cooperating tension device 20.

FIG. 9 illustrates yet a further modified practice of the invention, wherein a lateral compression device 21, such as that pictured in FIG. 8, is employed along with an adjustable vertical compression device 23 which is suitably positioned to act between the base of the material forming device 21 and the upper part of the forearm near the bent elbow condition. Such an additional compression device 23 is preferably, and in any suitable manner, made adjustable, as is indicated schematically in FIG. 9 at 23a, and when put into use, presses upwardly on the underside of the block of material forming compression device 21 to cause it to deform as is illustrated in a very exaggerated manner in FIG. 9 (see the solid outline of device 21 in comparison to the dashed outline thereof). Very clearly, changes in the amount of compression applied by device 23, which compression produces, through tension created in the humerus, a downward decompression force on the region of the rotator cuff, also acts to create and allow modifications in the lateral separation force produced through device 21.

Turning attention now to FIGS. 10 and 11 in the drawings, here there is illustrated still another apparatus form of the invention, wherein a downward decompression force, delivered through tension in the humerus to the region underlying and adjacent the torn rotator cuff, is created through an elongate compression device, such as that shown generally at 26. Such a device may take on a number of different forms, such as a spring-biased extension form, wherein an appropriate force adjuster, such as that shown schematically at 30, is provided to change the amount of compression force which it can exert through its opposite ends which are shown generally at 28 in FIG. 10. Preferably, device 26 acts along the side of the body between the underarm UA and the upper region of the forearm which is disposed just inwardly from the bent elbow condition BE.

It will thus be seen that the methodology of the present invention, implemented by any one or more of the various forms of force application devices which have been described and illustrated herein, produces, in all instances, a downward decompression force on the region adjacent an injured rotator cuff, and in some instances, both lateral and downward decompression forces relative to this region. These forces may be made to be adjustable, and it will be very clear that practice of the invention can be implemented not only when a person is sleeping and lying horizontal but at any time, inasmuch as the apparatus for implementing the decompression forces which are proposed is fully ambulatory with the person, and is substantially completely gravity-independent.

Practice of the invention definitively approaches addressing the pain and healing issues associated with a torn rotator cuff through furnishing very specifically created downward, and in some instances downward and lateral outward, decompression forces. Practice of the invention entails no expensive, difficult, and potentially problem-causing, invasive surgery, and also, definitively, does not rely upon rigid, nominal-condition stabilization and immobilizing of the rotator cuff area. Quite the contrary is involved because of the fact that the decompression forces proposed and implemented by the present invention positively shift relative positions between anatomical components in the shoulders so as to relieve normal-condition compression which would otherwise exist on and with respect to a rotator cuff tear.

Accordingly, while several preferred embodiments of the invention have been illustrated and described herein, it will be appreciated by those skilled in the art that the specific kinds of decompression forces proposed by the present invention can be created in a number of different ways utilizing various different kinds of force-applying, and preferably completely intra-anatomical, force-applying devices.

Those skilled in the art will also appreciate from expressions which are now given to describe generally the methodology of the present invention, that these method steps may be implemented in various forms and ways.

One manner of thinking about the practice of the present invention is to describe it as a method for addressing a torn rotator cuff including the practice of applying to the region of such a cuff, via the humerus which is associated with that cuff, an ambulatory, non-gravity-based, downwardly directed, elastic traction force. Such a force may, as has been explained and illustrated, be applied through the creation of either of an intra-anatomical tension force, or through the creation of an intra-anatomical compression force.

Another way of viewing the invention is to describe it as being a method of relieving compression pressure on an injured rotator cuff, including the steps of (a) applying to the upper arm which is associated with that cuff a gravity-independent, effective traction force, (b) maintaining that force for a user-selected time interval, and (c) by such force application, and during the user-selected time interval, decompressing the region of the injured cuff in a direction which is essentially along the long axis of the upper arm, or alternatively, both along the long axis of the upper arm and laterally outwardly.

Generally speaking, apparatus for practicing the invention includes (a) a subject-wearable, passive, yieldable-resistance, elongate, force-applying device having opposite ends, with that device, between its ends, possessing a line of action which lies generally parallel to the long axis of a subject's associated humerus, and (b) structure which is operatively associated with the opposite ends of the force-applying device for anchoring those ends effectively and operatively to spaced points on a subject's anatomy in manner whereby the force-applying device applies to the anatomy a force which tends to produce separation between the head of the humerus and the rotator cuff.

Accordingly, all forms of apparatus, and arrangements of method steps which effectively implement the concept of the invention, are deemed to come within the scope of the claims now presented herein.

I clain:

1. A method for addressing shoulder issues including each of (a) a torn rotator cuff, and (b) a post-surgery-shoulder-surgery condition, comprising applying to the region of such a rotator cuff/condition, via the associated humerus, an ambulatory, non-gravity-based, downwardly directed, elastic traction force.

2. The method of claim 1 which further comprises simultaneously applying, to the torn rotator cuff/condition region, a laterally outwardly directed force.

3. The method of claim 1, wherein said applying takes place through creating an intra-anatomical tension force.

4. The method of claim 3, wherein said creating is implemented via an elongate yieldable-resistance structure which extends and acts effectively between the elbow and the thigh.

5. The method of claim 4, wherein the mentioned elastomeric structure produces a selectable nominal tension force, and is action-length-adjustable to change that force.

6. The method of claim 4, wherein said creating is performed in relation to a bent-elbow condition.

7. The method of claim 1, wherein said applying takes place through creating an intra-anatomical compression force.

8. The method of claim 7, wherein said creating is implemented via an elongate, yieldable-resistance compression device designed to interact with the anatomy.

9. The method of claim 8, wherein the compression device produces a selectable, nominal compression force, and is adjustable to change the magnitude of that force.

10. The method of claim 8, wherein said creating is performed in relation to a bent-elbow condition.

11. Apparatus for addressing shoulder issues including each of (a) a subject's rotator cuff tear, and (b) a subject's post-shoulder-surgery condition comprising, at least,
a subject-wearable, passive, elongate, yieldable-resistance, force-applying device possessing opposite ends, and between said ends a line of action which, with the apparatus in place relative to such a subject and to the subject's rotator cuff/condition, lies generally parallel to the long axis of the subject's associated humerus, and
structure associated with said opposite ends for anchoring those ends operatively to spaced points on the subject's anatomy in a manner whereby the force-applying device applies to the anatomy a force which tends to produce separation between the head of the humerus and the rotator cuff.

12. The apparatus of claim 11, wherein said device is a tension device.

13. The apparatus of claim 12, wherein said device has one of its ends constructed for attachment to the anatomy generally at the location of the elbow which is associated with the subject's rotator cuff/condition, and its opposite end constructed for attachment to the anatomy generally at the location of the thigh which is generally directly below the rotator cuff tear/condition.

14. The apparatus of claim 11, wherein said device is a compression device.

15. A method of relieving compression pressure on the shoulder region which includes a rotator cuff including the steps of
applying to the upper arm which is associated with that cuff a gravity-independent, effective traction force,
maintaining that force for a user-selected time interval, and
by such force application, and during the user-selected time interval, decompressing the shoulder region which includes the rotator cuff in a direction which is essentially along the long axis of the upper arm.

16. The method of claim 15 which further comprises stabilized, lateral separation between the upper arm and the upper body.

17. The method of claim 15 which is effectively and preferably implemented through bending the forearm relative to the upper arm in order to create a generally right-angular relationship between the two, and by then introducing the desired traction force through suitable force application to the forearm near its region of adjacency with the angularly linked upper arm.
